(19) United States
(12) Patent Application Publication
(10) Pub. No.: US 2020/0001011 A1
(43) Pub. Date: Jan. 2, 2020

(54) PREFILLED SYRINGE AND PRESERVATION METHOD FOR RESIN COMPOSITE

(71) Applicant: NAMICS CORPORATION, Niigata (JP)

(72) Inventors: Hiroki MYODO, Niigata (JP); Toyokazu HOTCHI, Niigata (JP); Masaaki HOSHIYAMA, Niigata (JP)

(21) Appl. No.: 16/490,310

(22) PCT Filed: Mar. 6, 2018
(86) PCT No.: PCT/JP2018/008653

§ 371 Date: Aug. 30, 2019

(30) Foreign Application Priority Data
Mar. 13, 2017 (JP) 2017-047609

(51) Int. Cl. A61M 5/28; B05C 11/10; A61M 5/31

(52) U.S. Cl. CPC A61M 5/28; A61M 5/3129; B05C 11/10

(57) ABSTRACT

Provided are a filled syringe and a method for storing a resin composition, which can suppress generation of a void between the resin composition and an inner surface of the syringe. A filled syringe 1 includes a syringe 10, a plunger 20, and a resin composition 30. When a longitudinal direction of the syringe 10 is a vertical direction and the plunger 20 is above the resin composition 30, a ratio of a cross-sectional area 25 of the plunger at a position of a liquid level 31 at top of the resin composition to a cross-sectional area 35 enclosed by an inner surface 11 of the syringe at the same position as the liquid level 31 at the top of the resin composition is 95% or less.

### TECHNICAL FIELD

The present disclosure relates to a syringe filled with a resin composition and a method for storing the resin composition.

### BACKGROUND ART

Currently, a sealing resin composition may be filled in the syringe before being shipped, and then frozen and transported in some cases. Conventionally, the sealing resin composition has been filled so as not to contact air inside the syringe, when the plunger contacts a liquid level of the sealing resin composition (for example, PATENT LITERATURE 1).

However, in the conventional filling method, when a customer thaws the sealing resin composition before use, there is a problem that a gap is generated between an inner surface of the syringe and the resin composition to generate a void due to a contraction difference caused by a difference in thermal expansion coefficient between the resin composition and the syringe. Due to generation of the void, when using the syringe, problems occur such that discharging of the resin composition is interrupted, or a discharge amount of the resin composition is not stable.

### CITATION LIST

### Patent Literature

PATENT LITERATURE 1: JP-A-2012-045444

### SUMMARY OF THE INVENTION

### Problems to be Solved by the Invention

An object of the present disclosure is to provide the filled syringe and the method for storing the resin composition, which can suppress the generation of the void between the resin composition and the inner surface of the syringe.

### Solution to the Problems

The present inventors have found the following. That is, a space is provided between the plunger and the resin during storage. Air bubbles generated between the resin composition and the inner surface of the syringe, which are generated upon thawing, are moved to the space between the plunger and the resin. Thus, when using the resin composition, it is possible to prevent the air bubbles from remaining as the void between the resin composition and the inner surface of the syringe.

The present disclosure relates to the filled syringe and the method for storing the resin composition, in which the above problems are solved by having the following configuration.
[1] A filled syringe including a syringe, a plunger, and a resin composition, wherein when a longitudinal direction of the syringe is a vertical direction and the plunger is above the resin composition, a ratio of a cross-sectional area of the plunger at a position of a liquid level at top of the resin composition to a cross-sectional area enclosed by an inner surface of the syringe at the same position as the liquid level at the top of the resin composition is 95% or less.
[2] The filled syringe according to the above [1], wherein a temperature of the resin composition is −60 to 0° C.
[3] The filled syringe according to the above [1] or [2], wherein a distance between the liquid level at the top of the resin composition and a position in which a wiper of the plunger is in contact with the inner surface of the syringe is 0.2 mm or more.
[4] The filled syringe according to any one of the above [1] to [3], wherein the resin composition comprises at least one member of the group consisting of an epoxy resin and an acrylic resin.
[5] The filled syringe according to any one of the above [1] to [4], wherein the syringe is made of polypropylene or polyethylene.
[6] The filled syringe according to any one of the above [1] to [5], wherein a tip of the plunger is conical or flat.
[7] The filled syringe according to any one of the above [1] to [6], wherein an internal volume of the syringe is 3 cm³, 5 cm³, 10 cm³, 30 cm³, 50 cm³, 55 cm³ or more.
[8] A method for storing a resin composition using the filled syringe according to any one of the above [1] to [7].

### Effects of the Invention

According to the present disclosure [1], it is possible to provide the filled syringe which can suppress the generation of the void between the resin composition and the inner surface of the syringe.

According to the present disclosure [8], it is possible to provide the method for storing the resin composition, which can suppress the generation of the void during storage.

### BRIEF DESCRIPTION OF THE DRAWINGS

FIG. 1 shows an example of a conceptual view of a cross-section of a filled syringe of the present disclosure.

FIG. 2 shows an example of a plunger included in the filled syringe of the present disclosure.

### DESCRIPTION OF THE EMBODIMENTS

The filled syringe of the present disclosure (hereinafter referred to as “filled syringe”) includes a syringe, a plunger, and a resin composition. When the longitudinal direction of this syringe is a vertical direction and the plunger in the syringe is above the resin composition, a ratio of a cross-sectional area of the plunger (hereinafter referred to as “plunger cross-sectional area” as appropriate) at a position of a liquid level at top of the resin composition to a cross-sectional area enclosed by an inner surface of the syringe (hereinafter referred to as “syringe cross-sectional area” as appropriate) at the same position as the liquid level at the top of the resin composition is 95% or less.

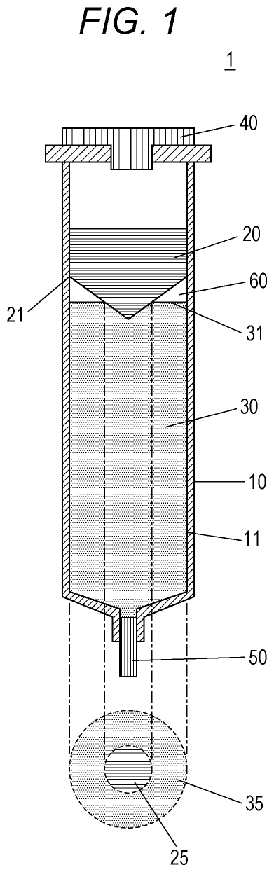

FIG. 1 shows an example of a conceptual view of a cross-section of a filled syringe of the present disclosure. Hereinafter, it will be described with reference to FIG. 1. As shown in FIG. 1, a filled syringe 1 of the present disclosure includes a syringe 10, a plunger 20, and a resin composition 30. When the longitudinal direction of the syringe 10 is the vertical direction and the plunger 20 is above the resin composition 30, the ratio of a plunger cross-sectional area 25 of the plunger 20 at the position of a liquid level 31 at the top of the resin composition to a syringe cross-sectional area 35 enclosed by an inner surface 11 of the syringe 10 at the same position as the liquid level 31 at the top of the resin composition is 95% or less.

As can be seen from FIG. 1, in the filled syringe 1, a space 60 is present between the plunger 20 in the syringe 10 and the liquid level 31 at the top of the resin composition 30. By expanding a volume of the space 60 to a specific amount or more, it is possible to suppress generation of air bubbles when the resin composition 30 is thawed. That is, even if the air bubbles are generated between the inner surface 11 of the syringe and the resin composition 30, the air bubbles move to the space 60.

When using the filled syringe, after returning a temperature of the filled syringe 1 to room temperature, a head cap 40 is removed and the plunger 20 is pushed downward by a rod (not shown). In this manner, air can be discharged upwardly of the plunger 20 by bringing the plunger 20 into contact with the liquid level 31 at the top of the resin composition. Therefore, it is possible to prevent discharge of the resin composition from being interrupted when using the filled syringe. As a result, a discharge amount of the resin composition can be stabilized. In addition, a needle cap 50 can be removed to attach a needle (not shown in FIG. 1) as necessary.

Here, the plunger 20 is not particularly limited as long as it has a function of pushing the resin composition 30 in the syringe 10 out to the needle side. The plunger 20 may also be referred to as a piston or the like.

Note that when an interface at the top of the resin composition 30 has a fillet, a position of the interface except the fillet is the liquid level 31 at the top of the resin composition 30.

The temperature of the filled syringe 1 is preferably −60 to 0° C., more preferably −20° C. or less, and still more preferably −40° C. or less, from the viewpoint of ease of exhibiting effects of the present disclosure.

Similarly, from the viewpoint of the ease of exhibiting the effects of the present disclosure, a distance between the liquid level 31 at the top of the resin composition 30 and a position in which a wiper 21 of the plunger 20 contacts the inner surface 11 of the syringe 10 is preferably 0.2 mm or more, and more preferably 1 mm or more. Here, the wiper 21 refers to a tip portion (lower end portion in FIG. 1) of the plunger 20.

Characteristics of the resin composition 30 are not particularly limited. For example, the resin composition 30 may be insulating or conductive. However, the resin composition containing at least one member of the group consisting of an epoxy resin and an acrylic resin is preferable because the effects of the present disclosure are easily exhibited.

A material of the syringe 10 is not particularly limited. However, from the viewpoint of durability of the syringe, the syringe 10 is preferably made of polypropylene or polyethylene, and more preferably polypropylene. An internal volume of the syringe is 3 cm³, 5 cm³, 10 cm³, 30 cm³, 50 cm³, 55 cm³ or more from the viewpoint of the ease of exhibiting the effects of the present disclosure.

The material of the plunger 20 is preferably PP or PE from the viewpoint of durability. The tip portion (lower end portion in FIG. 1) of the plunger 20 is preferably conical or flat from the viewpoint of extrudability of the resin composition 30.

By using the above-described filled syringe and storing the resin composition, it is possible to suppress the generation of the void during storage.

### EXAMPLES

The present embodiment will be described by way of Examples. However, the present embodiment is not limited to the Examples. Note that in the following examples, parts and % indicate parts by mass and % by mass unless otherwise specified.

### [Preparation of Resin Composition]

In 41.9 parts by mass of liquid acrylic resin (product name: A-DCP) manufactured by Shin-Nakamura Chemical Co., Ltd., 58 parts by mass of silica filler was dispersed by a roll mill. Then, 0.1 parts by mass of an organic peroxide as a curing agent was blended to the obtained mixture of the acrylic resin and the silica filler.

### [Preparation of Filled Syringe]

The syringe was filled with the resin composition under conditions shown in Tables 1 and 2. The plunger was then inserted into the syringe from above. In this way, the filled syringe was prepared. Tables 1 and 2 show syringe material, syringe size, plunger type, filling amount, storage temperature, liquid level-space area, and liquid level-plunger height used. Here, as shown in FIG. 1, the liquid level-space area is a ratio (unit: %) of a difference between the syringe cross-sectional area 35 and the plunger cross-sectional area 25 to the syringe cross-sectional area 35. As shown in FIG. 1, the liquid level-plunger height is the distance between the liquid level 31 at the top of the resin composition 30 and the position in which the wiper 21 of the plunger 25 contacts the inner surface 11 of the syringe 10. A syringe manufactured by EFD (Optimum series, mainly 30 cm³) was used as the syringe. Here, as the syringe material, PP shows polypropylene and PE shows polyethylene. The liquid level-plunger height was measured by a measuring microscope manufactured by Olympus. The tip of the plunger used in Examples 7 and 8 described in Table 2 was substantially flat. Thus, almost no inclination was observed between an outer periphery and a central portion of the plunger. Therefore, the liquid level-space area could not be calculated. However, the space was present between the liquid level at the top of the resin composition and the plunger. The height between the liquid level and the plunger was 1 mm. From this, it was estimated that the ratio of the cross-sectional area of the plunger to the cross-sectional area of the syringe was 95% or less.

### [Void Evaluation]

At the storage temperature shown in Tables 1 and 2, the filled syringe was kept frozen for 4 hours or more. The filled syringe kept frozen was kept at room temperature for 1 hour. Thereafter, presence or absence of voids in the filled syringe was evaluated by visual observation. Tables 1 and 2 show the results.

### [Evaluation of Discharge Interruption]

At the storage temperature shown in Tables 1 and 2, the filled syringe was kept frozen for 4 hours or more. The filled syringe kept frozen was kept at room temperature for 1 hour. Thereafter, the plunger was pushed in, and a 25G needle was attached to the filled syringe. A line of the resin composition was drawn until all the resin composition inside the syringe was discharged using a table-top type liquid agent application robot 2000N series manufactured by SAN-EI TECH Ltd. The presence or absence of interruption of the line was evaluated by visual observation. Tables 1 and 2 show the results.

TABLE 1

| | Comparative | Comparative | Comparative | Comparative | | | | | | |
|---|---|---|---|---|---|---|---|---|---|---|
| | Example 1 | Example 2 | Example 3 | Example 4 | Example 1 | Example 2 | Example 3 | Example 4 | Example 5 | Example 6 |
| Syringe material | PP | PP | PP | PP | PP | PP | PP | PP | PP | PP |
| Syringe size | 30 | 30 | 30 | 30 | 30 | 30 | 30 | 30 | 30 | 30 |
| (unit: cm³) | | | | | | | | | | |
| Plunger type¹⁾ | 1 | 1 | 1 | 1 | 1 | 1 | 1 | 1 | 1 | 1 |
| filling amount | 10 | 10 | 10 | 10 | 10 | 10 | 10 | 10 | 10 | 5 |
| (unit: cm³) | | | | | | | | | | |
| Storage | −40 | −40 | −40 | −20 | −40 | −20 | −50 | −40 | −40 | −40 |
| temperature | | | | | | | | | | |
| (unit: ° C.) | | | | | | | | | | |
| liquid level-space | 0 | 3 | 4 | 4 | 5 | 5 | 5 | 10 | 100 | 10 |
| area (unit: %) | | | | | | | | | | |
| liquid | 0 | 0.12 | 0.16 | 0.16 | 0.20 | 0.20 | 0.20 | 0.42 | 6.00 | 0.42 |
| level-plunger | | | | | | | | | | |
| height | | | | | | | | | | |
| (unit: mm) | | | | | | | | | | |
| Void | Yes | Yes | Yes | No | No | No | No | No | No | No |
| Discharge | Yes | Yes | Yes | Yes | No | No | No | No | No | No |
| interruption | | | | | | | | | | |

1) Plunger type

1: 5112PE 30CC/55CC NO-DRIP WIPER PISTON (EFD)

TABLE 2

| | | | | Example | Example | Example | Example | Example | Example |
|---|---|---|---|---|---|---|---|---|---|
| | Example 7 | Example 8 | Example 9 | 10 | 11 | 12 | 13 | 14 | 15 |
| Syringe material | PE | PE | PP | PP | PP | PP | PP | PP | PP |
| Syringe size (unit: cm³) | 180 | 360 | 10 | 55 | 30 | 30 | 30 | 30 | 30 |
| Plunger type¹⁾ | 3 | 3 | 4 | 1 | 1 | 1 | 1 | 2 | 2 |
| filling amount (unit: cm³) | 80 | 200 | 10 | 10 | 10 | 10 | 10 | 10 | 10 |
| Storage temperature | −40 | −40 | −40 | −40 | −40 | −40 | −40 | −40 | −40 |
| (unit: ° C.) | | | | | | | | | |
| liquid level-space area | Unable to | Unable to | 5 | 5 | 25 | 100 | 100 | 9 | 9 |
| (unit: %) | calculate | calculate | | | | | | | |
| liquid level-plunger | 1.00 | 1.00 | 0.13 | 0.20 | 1.00 | 5.00 | 10.00 | 1.00 | 10.00 |
| height (unit: mm) | | | | | | | | | |
| Void | No | No | No | No | No | No | No | No | No |
| Discharge interruption | No | No | No | No | No | No | No | No | No |

1) Plunger type

1: 5112PE 30CC/55CC NO-DRIP WIPER PISTON (EFD)

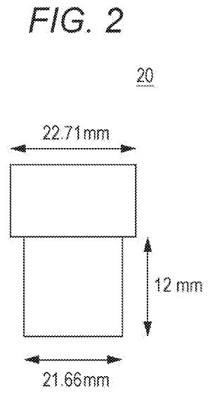

2: In-house produced plunger shown in FIG. 2

3: 5196 PRS CARTRIDGE PLUNGER WITH SKIRT (produced by SEMCO)

4: 5111PE 10 cc SMOOTH FLOW WIPER PISTON (EFD)

As can be seen from Tables 1 and 2, neither void generation nor discharge interruption was observed in all of Examples 1 to 15. In contrast, the void was generated and the discharge was interrupted in Comparative Example 1. In Comparative Example 1, the liquid level-space area is 0% (the ratio of the plunger cross-sectional area 25 to the syringe cross-sectional area 35 in FIG. 1 is 100% (the same applies to Comparative Examples 2 to 4)). Similarly, the void was generated and the discharge was interrupted also in Comparative Example 2. In Comparative Example 2, the liquid level-space area is 3% (the ratio of the plunger cross-sectional area 25 in FIG. 1 is 97%). The void was generated and the discharge was interrupted also in Comparative Example 3. In Comparative Example 3, the liquid level-space area is 4% (the ratio of the plunger cross-sectional area 25 in FIG. 1 is 96%). The discharge was interrupted in Comparative Example 4. In Comparative Example 4, the liquid level-space area is 4% (the ratio of the plunger cross-sectional area 25 in FIG. 1 is 96%). A difference between Comparative Example 3 and Comparative Example 4 is the storage temperature. In Comparative Example 3, the discharge was interrupted and the void was generated in the resin composition stored at a lower temperature. Although not described in Table 1, similar results were obtained even when using the resin composition containing a liquid epoxy resin, a curing agent (at least one selected from the group consisting of amine-based, phenol-based, and acid anhydride-based curing agents), and a dispersed silica filler as the resin composition.

As described above, according to the present disclosure, it is possible to provide the filled syringe and the method for storing the resin composition, which can suppress the generation of the void between the resin composition and the inner surface of the syringe.

### LIST OF REFERENCE NUMERALS

1: Filled syringe, 10: Syringe, 11: Inner surface of syringe, 20: Plunger, 21: Wiper of plunger, 25: Plunger cross-sectional area at the same position as liquid level 31 at top of resin composition, 30: Resin composition, 31: Liquid level at top of resin composition, 35: Cross-sectional area of inner surface 11 of syringe at position of liquid level 31 at top of resin composition, 40: Head cap, 50: Needle cap, 60: Space.

1. A filled syringe comprising a syringe, a plunger, and a resin composition, wherein
when a longitudinal direction of the syringe is a vertical direction and the plunger is above the resin composition, a ratio of a cross-sectional area of the plunger at a position of a liquid level at top of the resin composition to a cross-sectional area enclosed by an inner surface of the syringe at the same position as the liquid level at the top of the resin composition is 95% or less.

2. The filled syringe according to claim 1, wherein
a temperature of the resin composition is −60 to 0° C.

3. The filled syringe according to claim 1, wherein
a distance between the liquid level at the top of the resin composition and a position in which a wiper of the plunger is in contact with the inner surface of the syringe is 0.2 mm or more.

4. The filled syringe according to claim 1, wherein
the resin composition comprises at least one member of the group consisting of an epoxy resin and an acrylic resin.

5. The filled syringe according to claim 1, wherein
the syringe is made of polypropylene or polyethylene.

6. The filled syringe according to claim 1, wherein
a tip of the plunger is conical or flat.

7. The filled syringe according to claim 1, wherein
an internal volume of the syringe is 3 cm³, 5 cm³, 10 cm³, 30 cm³, 50 cm³, 55 cm³ or more.

8. A method for storing a resin composition using the filled syringe according to claim 1.
